(19) United States
(12) Patent Application Publication
(10) Pub. No.: US 2026/0269081 A1
(43) Pub. Date: Sep. 10, 2026

(54) Distributed System for Identifying the Appropriate Heart Valve Implant Using an Ensemble Machine-Learning Algorithm, Synthetic Data, and Edge Computing

(71) Applicant: Anaclara Systems Limited, Little Kimble (GB)

(72) Inventors: Andrew DEONARINE, New York, NY (US); William Railton FRITH, Little Kimble (GB); Victor NORDBERG, Plymouth, MA (US)

(21) Appl. No.: 19/560,846

(22) Filed: Mar. 9, 2026

Related U.S. Application Data
(60) Provisional application No. 63/768,774, filed on Mar. 7, 2025.

(51) Int. Cl. G16H 50/70; G06N 5/01; G06N 20/20; G16H 50/30

(52) U.S. Cl. CPC G16H 50/70; G06N 5/01; G06N 20/20; G16H 50/30

(57) ABSTRACT

A system and method for and method for selecting an implantable heart valve for patients using is disclosed. The system includes analyzing population-level environmental exposure data to categorize geographic locations by cardiovascular risk. A synthetic clinical dataset is generated using a generative adversarial network along with predefined machine-learning parameters. The user device calculates an exposomic feature value by weighting pollutant concentrations based on residence durations. The user device is then trained using a machine-learning model, selected from random forest models, gradient-boosted decision tree models, support vector machines, artificial neural networks, and elastic net regression. After training, the model is executed on the user device using an edge computing approach with patient-specific data and exposomic feature values to generate heart valve intervention options that minimize the difference between the predicted remaining lifespan of the patient and the valve's operational lifespan while reducing the risk of failure or complications.

### CROSS-REFERENCE TO RELATED APPLICATIONS

This patent application claims the benefit of U.S. Patent Application No. 63/768,774, filed March 7, 2025, and the entire contents of which are incorporated herein by reference.

### BACKGROUND

Unless otherwise indicated herein, the materials described in this section are not prior art to the claims in this application and are not admitted to being prior art by inclusion in this section.

### Technical Field

The present disclosure generally relates to computer-implemented medical technologies, and more particularly relates to systems and methods for selecting an implantable heart valve for a patient using patient-specific data and medical imaging data processed by a distributed calculating system.

### Discussion of Prior Art

Every year, hundreds of thousands of people undergo heart valve replacement procedures worldwide. Patients may require heart valve replacement due to conditions such as valve regurgitation or valve stenosis, which can arise from diseases including rheumatic heart disease, endocarditis, or age-related degeneration. Common heart valve replacement procedures include transcatheter aortic valve replacement (TAVR), surgical aortic valve replacement (SAVR), and transcatheter mitral valve replacement (TMVR). Replacement valves may include mechanical valves, tissue-based valves, or valves derived from a patient’s own tissue, such as in the Ross procedure.

The selection of an implantable heart valve is a critical clinical decision, as the chosen valve must properly fit the patient’s anatomy and provide reliable function over time. An improperly selected valve may result in reduced durability, complications, or the need for additional surgical intervention, which can increase patient risk and negatively impact long-term health outcomes. Conventionally, the selection of an implantable heart valve for a patient is performed using manual or partially manual clinical processes. These processes typically involve clinician review of patient-specific information, such as medical imaging, anatomical measurements, and diagnostic reports. The clinician compares this information to manufacturer guidelines, valve sizing charts, and prior procedural experience to determine a suitable heart valve.

In these conventional approaches, the basis for selecting a particular heart valve is often not generated or recorded in a structured or consistent manner. The decision may depend largely on individual clinical judgment, which can vary between clinicians and healthcare institutions. As a result, valve selection decisions may be difficult to reproduce, validate, or explain in a consistent way. Additionally, heart valves selected using existing methods may not be optimized for the patient’s long-term health. In some cases, a selected valve may have a shorter functional lifespan than the patient’s remaining life expectancy, increasing the likelihood of future reoperation or complications. These risks are especially significant for older patients and patients with additional medical conditions. While some software tools exist to assist clinicians in valve selection, such tools are typically limited to basic rule-based recommendations and do not fully integrate patient-specific data or clearly explain how individual patient characteristics influence the selection outcome

In light of the foregoing, there is a need for a system and method that improves the selection of implantable heart valves by providing more consistent, data-driven, and interpretable decision support, thereby reducing clinician uncertainty and helping ensure that the selected valve is appropriate for both the patient’s anatomy and long-term health needs.

### SUMMARY

In an example embodiment, a system and method for selecting an implantable heart valve for patients using a distributed computing system is disclosed. The system comprises a computer server including processors and a memory module. The processor is configured to analyze population-level environmental exposure data, such as atmospheric pollutant concentrations, to categorize geographic locations by cardiovascular risk. The processor is further configured to generate a synthetic clinical dataset using a generative adversarial network, producing patient data based on exposure-based geographic and valve prosthesis categories. This dataset, along with predefined machine-learning parameters, is sent to a user device along with patient-specific data, including clinical parameters and a longitudinal residence history, which tracks geographic locations and residence durations. The user device calculates an exposomic feature value by weighting pollutant concentrations based on residence durations. The user device is then trained an tested using a supervised ensemble machine-learning model, selected from gradient-boosted decision tree models and random forest models, with the synthetic dataset and parameters. After training, the model is executed on the user device with patient-specific data and exposomic feature values to generate implantable heart valve options that minimize the difference between the predicted remaining lifespan of the patient and the valve's operational lifespan while reducing the risk of failure or complications.

### BRIEF DESCRIPTION OF DRAWINGS

Example embodiments are illustrated by way of example and not limitation in the figures of the accompanying drawings, in which like references indicate similar elements and in which:

FIG. 1 illustrates a schematic overview of a system 100 for selecting an implantable heart valve for patients, in accordance with an example embodiment;

FIG. 2 illustrates an exemplary process for extracting and processing features 206-216 from individual-level data and population-level data, in accordance with the example embodiment;

FIG. 3 illustrates a pollutant analysis system 300 integrated with system 100, in accordance with the example embodiment;

FIG. 4 illustrates a system 400 for generating a synthetic clinical dataset 408 using a CGAN, in accordance with the example embodiment;

FIG. 5 illustrates an ensemble machine-learning process 500 for evaluating patient-valve compatibility, valve lifespan, and predicted risk outcomes, in accordance with the example embodiment;

FIG. 6A illustrates a method 600 for selecting an implantable heart valve for a patient, in accordance with an example embodiment;

FIG. 6B illustrates an example execution of a machine-learning model trained and tested as an edge computing workflow on a user device 104, in accordance with an example embodiment;

FIG. 7 illustrates a computing environment 700 for implementing system 100 for selecting implantable heart valves for patients, in accordance with the example embodiment; and

FIG. 8 illustrates the hardware implementation of the server 110 on which the system 100 is executed, in accordance with the example embodiment.

### DETAILED DESCRIPTION

The following detailed description includes references to the accompanying drawings, which form a part of the detailed description. The drawings show illustrations in accordance with example embodiments. These example embodiments, which may be herein also referred to as “examples” are described in enough detail to enable those skilled in the art to practice the present subject matter. However, it may be apparent to one with ordinary skill in the art, that the present subject matter may be practiced without these specific details. In other instances, well-known methods, procedures, and components have not been described in detail so as not to unnecessarily obscure aspects of the example embodiments. The example embodiments can be combined, other example embodiments can be utilized, or structural, logical, and design changes can be made without departing from the scope of the claims. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive “or” such that “A or B” includes “A but not B,” “B but not A,” and “A and B,” unless otherwise indicated.

Reference in the specification to “one embodiment” or to “an embodiment” means that a particular feature, structure, or characteristic described in connection with the embodiments is included in at least one embodiment of the system and method, and multiple references to “one embodiment” or “an embodiment” should not be understood as necessarily all referring to the same embodiment.

The selection of the appropriate heart valve for a patient is a complex and critical task that requires a detailed assessment of various factors, including the patient’s anatomical structure, age, medical history, and the nature of their heart condition. With advancements in medical technology, there is an increasing need for a heart valve that not only meets the immediate medical requirements of the patient but also offers long-term durability. The chosen valve must be able to withstand the patient’s unique physiological demands throughout their life, providing reliable performance over time. Ensuring the valve’s longevity is vital, as it should last slightly longer than the patient’s own lifespan, adapting to evolving health needs without compromising their well-being.

In light of the foregoing, there is a need for a system and method capable of enhancing the heart valve selection process by improving its accuracy, consistency, and interpretability. Additionally, it would reduce clinician uncertainty, enabling the selection of the heart valve that aligns both with the patient’s anatomical needs and long-term health considerations, thereby improving patient outcomes and reducing the risk of complications or valve failure.

In an example embodiment, a system and method for selecting an implantable heart valve for patients using a distributed computing system is disclosed. The system includes a server with processors that analyze population-level environmental exposure data, such as atmospheric pollutant concentrations, to categorize geographic locations by cardiovascular and general health risk. The server generates a synthetic clinical dataset using a generative adversarial network (GAN such as a conditional tabular generative adversarial network (CTGAN)), producing patient data based on exposure-based geographic and valve prosthesis categories. This dataset, along with predefined machine-learning parameters, is sent to a user device along with patient-specific data, including clinical parameters and a longitudinal residence history, which tracks geographic locations and residence durations. The user device calculates an exposomic feature value by weighting pollutant concentrations based on residence durations. The user device is then trained using a supervised ensemble machine-learning model, selected from gradient-boosted decision tree models and random forest models, with the synthetic dataset and parameters. After training, the model is executed on the user device with patient-specific data and exposomic feature values to generate implantable heart valve options that minimize the difference between the predicted remaining lifespan of the patient and the valve's operational lifespan while reducing the risk of failure or complications.

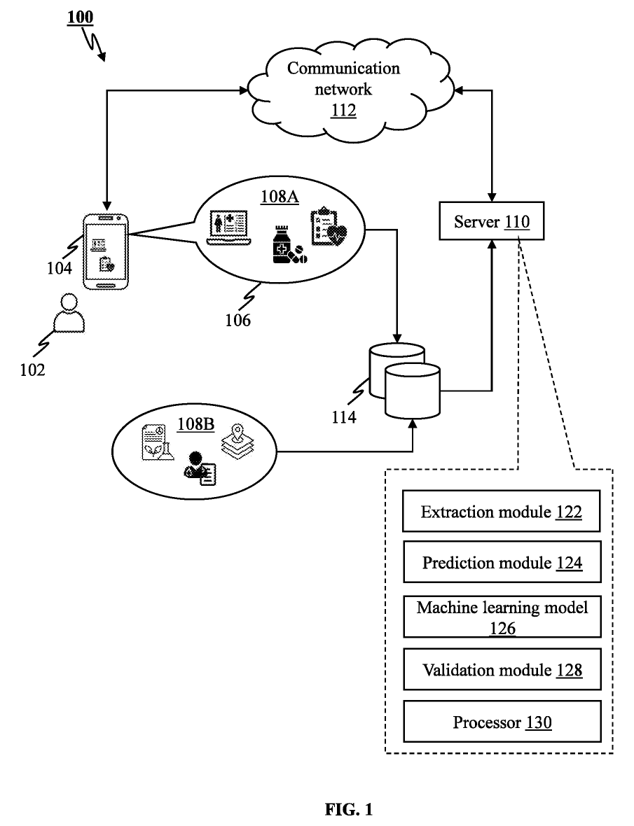

Referring to FIG. 1, in an example embodiment, a system 100 for selecting an implantable heart valve for a patient using a distributed computing system is disclosed. The system 100 comprises a user 102 interacting with an application platform 106 via a user device 104. The user 102 (also referred to as “patient”) may also be a clinician, a healthcare provider, or any other authorized individual seeking heart valve selection support. The user device 104 may be any computing device capable of executing software and communicating over the communication network 112, including but not limited to a smartphone, personal computer, laptop, tablet, workstation, or monitor. By operating the application platform locally on such hardware, the user device 104 can function as an edge computing node, allowing for secure, real-time data processing and decision support at the point of care. The application platform 106 may include a web-based application, a mobile application, a desktop application, or any combination thereof, and may be configured to securely receive, process, and transmit patient health information 108A associated with the user 102.

In an example embodiment, the system 100 may be configured to securely receive, process, and transmit patient health information 108A and related clinical data 108B. The patient health information 108A may include a longitudinal residence history, which may include, but is not limited to, a chronological listing of multiple geographic locations and corresponding residence durations. These geographic locations may be employed to determine historical environmental exposure levels, which may be critical for establishing a comprehensive exposomic profile of the patient. The exposomic profile may be a longitudinal record of all environmental exposures an individual encounters throughout their life, which may include chemical, physical, and biological stressors that interact with the individual’s biology to influence health outcomes.

In an example embodiment, the patient health information 108A may further include, but is not limited to, individual health records comprising health indicators, body weight, age, sex, cardiovascular and heart-related diagnoses, prior medical procedures, diagnostic imaging data (e.g., echocardiograms or CT scans), laboratory results, and other physiological or clinical parameters relevant to heart valve selection. To supplement this individual data, the system 100 may employ related clinical data 108B, which may include data generated by clinicians to represent real patient populations, as well as simulated data comprising individual-level and population-level datasets. This population-level data may comprise social determinants of health (SDoH), such as unemployment rates, homelessness rates, income levels, education levels, and access to healthcare services. These variables may be associated with geographic units including, but not limited to, zip codes, census tracts, counties, or other defined geographical regions corresponding to where an individual resides. By integrating these SDoH factors, the system 100 may better account for systemic influences on patient recovery and long-term valve viability.

In an example embodiment, the related clinical data 108B may include exposome data capturing environmental, lifestyle, and external exposure factors at various geospatial levels. Such exposome data may include, but is not limited to, population-weighted atmospheric pollutant concentration values (eg., NO₂ or O₃ levels), air quality metrics, pollution exposure measures, climate-related variables (e.g., extreme temperature fluctuations), and other environmental risk factors.

In an example embodiment, both the patient health information 108A and the related clinical data 108B may be stored in a database 114 communicatively coupled to a server 110. The database 114 may be configured to store the patient health information 108A and the related clinical data 108B in a structured, unstructured, or tabular format. In addition to individual records, the database 114 may store population-level environmental exposure data. The population-level environmental exposure data may be a curated dataset comprising atmospheric and environmental metrics aggregated across specific demographics or geographic regions, which may include, but is not limited to, population-weighted atmospheric pollutant concentration values, air quality index (AQI) historical logs, and localized climate stressors. The data stored within the database 114 may include, but is not limited to, patient identifiers, age, gender, demographic attributes, and diagnosis codes, as well as metadata related to assigned or recommended cardiac valve implants. Furthermore, the database 114 may maintain records of estimated patient remaining lifespan, projected heart valve operational lifespan, and historical or predicted heart failure and complication rates. By maintaining the population-level environmental exposure data in a structured format alongside individual health records, the database 114 may enable the server 110 to perform high-velocity correlation between a patient’s residence history and the environmental risk factors inherent to those specific geographic locations.

The server 110 may be configured to access and process the data stored in the database 114, perform analytical and computational operations thereon, and generate output data used by the system 100 to assist in selecting an implantable heart valve that is suitable, compatible or even optimal for a given patient. The server 110 may further be communicatively coupled to the user device 104 via the communication network 112. The communication network 112 may include, but is not limited to, a local area network (LAN), wide area network (WAN), cellular network, wireless network, the Internet, or any combination of wired and wireless communication infrastructures, and may enable secure bidirectional data communication between the server 110 and the user device 104.

In an example embodiment, the server 110 may include one or more processors 130 communicatively coupled to an extraction module 122. The extraction module 122 may be configured to extract, normalize, and preprocess features from the patient health information 108A and the related clinical data 108B. The extracted features may include structured and unstructured attributes such as clinical measurements, diagnostic codes, and temporal variables. Further, the extraction module 122 may enable the performance of unsupervised machine-learning analysis on population-level environmental exposure data. This analysis may group geographic locations into exposure-based categories associated with cardiovascular risk, which may then be assigned unique geographic exposure identifiers. These identifiers may be linked to geographic locations in the population-level data to refine the patient's exposomic profile. Based on the extracted features and categories, the system 100 may be configured to predict one or more valve matches using a machine learning model 126.

In an alternate example embodiment, the performance of the system 100 may be evaluated and optimized using statistical performance metrics. These metrics may include, but are not limited to, precision (the ratio of correctly predicted positive observations to the total predicted positives), recall (the ratio of correctly predicted positive observations to all observations in the actual class), and the F1 score (the harmonic mean of precision and recall). Furthermore, the system 100 may employ Receiver Operating Characteristic (ROC) curves to visualize the trade-off between the true positive rate and false positive rate. Such evaluation measures may ensure that the predictions generated by a prediction module 124 maintain a high degree of clinical reliability.

In an example embodiment, the server 110 may include the prediction module 124 operably and communicatively coupled to the machine learning model 126. The prediction module 124 may be configured to serve as the primary analytical engine for synthesizing multi-dimensional inputs that includes, but not limited to, clinical, demographic, and exposomic data: into actionable, patient-specific outputs. The prediction module 124 may be configured to execute a comparative analysis between the patient’s unique physiological profile and a plurality of available heart valve prosthesis categories. Further, the prediction module 124 may generate predictive outputs that may include, but are not limited to, a predicted patient remaining lifespan and a predicted valve operational lifespan. The predicted patient remaining lifespan may refer to an estimated duration of the patient’s life based on the integration of individual health indicators and the cumulative impact of environmental stressors identified in the exposomic profile. Conversely, the predicted valve operational lifespan may refer to an estimation of the functional durability of a specific heart valve implant, accounting for biological degradation factors and the environmental context of the patient’s residence history.

In an alternate example embodiment, the prediction module 124 may apply a selection criterion to identify optimal valve matches. This selection criterion may include calculating a lifespan difference value, wherein the prediction module 124 seeks to minimize the discrepancy between the patient’s estimated longevity and the valve’s projected functionality. By optimizing for a reduced difference in these lifespans, the prediction module 124 may identify a valve that is neither prone to premature failure (which may necessitate high-risk re-intervention) nor excessively durable relative to the patient’s needs. Further, the prediction module 124 may be configured to calculate a predicted valve failure or complication probability. This probability may represent the statistical likelihood of adverse clinical events, such as structural valve deterioration or thrombosis, over a predefined temporal horizon. The resulting output from the prediction module 124 may include an ordered or ranked list of valve category identifiers, potentially annotated with statistical confidence parameters and risk estimates, which may be utilized by the clinician to facilitate an informed, individualized implantation strategy.

In an example embodiment, the predicted values generated by the prediction module 124 may subsequently be processed by a validation module 128. The validation module 128 may be configured to validate the predicted outputs based on predefined clinical thresholds (e.g., minimum acceptable valve durability) or confidence scores (e.g., a statistical measure of the model's certainty). Once validated, the outputs may be encoded, hashed, or encrypted using cryptographic techniques, such as Advanced Encryption Standard (AES) or Secure Hash Algorithms (SHA). This processing may ensure data integrity and confidentiality during transmission to the user device 104.

In an example embodiment, the user 102 may input the patient health information 108A and related clinical data 108B into the user device 104. The user device 104 may process the received data to compute structured clinical and exposomic feature values, which may then be provided as inputs to a trained and validated machine-learning model 126 stored locally on the user device 104. The machine-learning model 126 may comprise a multi-stage analytical structure that begins with an unsupervised machine-include analysis. The unsupervised machine-learning analysis may refer to a computational process where the model identifies latent patterns or clusters in a population-level environmental exposure dataset without the need for pre-labeled outcomes. For example, the system 100 may autonomously group various zip codes or census tracts into exposure-based categories by identifying shared environmental signatures, such as high-density urban areas with similar concentrations of nitrogen dioxide and ozone, which may be associated with increased cardiovascular risk.

Furthermore, the machine-learning model 126 may further include a trained ensemble machine-learning model that has been optimized using a synthetic clinical dataset and a predefined set of machine-learning parameters. The synthetic clinical dataset may refer to a non-real, machine-generated dataset, such as one produced by a Generative Adversarial Network (GAN such as a conditional tabular GAN), that maintains the statistical distribution and correlations of actual patient populations to enable robust training without compromising individual privacy. The predefined set of machine-learning parameters may refer to a collection of configuration variables, such as learning rates, tree depth, or number of estimators, that govern the training process and architecture of the model. Further, the trained ensemble machine-learning model may refer to a supervised learning structure that combines the predictive capacity of multiple constituent models to achieve superior accuracy and stability. In an example embodiment, the ensemble model may be selected from gradient-boosted decision tree models, which may be an iterative technique where new trees are sequentially added to correct errors made by previous trees, or random forest models, which may be a method that constructs a vast multitude of independent decision trees to output a consensus-based prediction.

In an example embodiment, the user device 104 may execute the trained ensemble machine-learning model (e.g., random forest models, gradient-boosted decision tree models, support vector machines, artificial neural networks, or elastic net regression) by including a uniquely calculated exposomic feature value. This exposomic feature value may be derived by the user device 104 by applying a residence-duration-based weighting to pollutant concentration values. This weighting ensures that the trained ensemble machine-learning model accounts for the specific length of time a patient resided in a given environment. For instance, a patient residing in an exposure-based category with high pollution for twenty years may be assigned a higher risk weight by the trained ensemble machine-learning model than a patient who resided in the same category for only two years.

By processing these comprehensive inputs, the trained ensemble machine-learning model may generate an output representing one or more implantable heart valves that satisfy a selection criterion. This criterion may involve identifying a reduced difference between a predicted remaining lifespan of the patient and a predicted operational lifespan of the heart valve. Simultaneously, the trained ensemble machine-learning model may ensure a reduced predicted valve failure or complication probability by analyzing the interactions between the patient’s longitudinal environmental context and the specific material properties of the heart valve category. Through this calculation, the trained ensemble machine-learning model may move beyond general clinical averages to provide a localized, patient-specific recommendation. For example, the trained ensemble machine-learning model may determine that a patient with high cumulative exposure to specific particulate matter requires a valve type with a higher resistance to accelerated calcification, thereby harmonizing the valve's durability with the patient’s projected health trajectory.

In an example embodiment, a clinician may input patient-specific information into the user device 104 for a 72-year-old male patient diagnosed with severe aortic stenosis, having a left ventricular ejection fraction of 48% and a valve annulus diameter of 23 mm, along with comorbid conditions including hypertension and chronic kidney disease. Based on the patient’s longitudinal residence history, the system 100 may further determine an elevated time-weighted aldehyde exposure level. When this patient-specific clinical and exposomic data is provided to the trained ensemble machine-learning model, the ensemble machine-learning model may predict a remaining patient lifespan of approximately fourteen years and evaluate multiple candidate implantable heart valves. The trained ensemble machine-learning model may determine that a transcatheter bioprosthetic aortic valve provides the closest alignment between the predicted patient lifespan and the predicted operational lifespan of the valve, while also exhibiting a lower predicted risk of valve failure or post-procedural complications compared to alternative surgical valve options. Accordingly, the system 100 may recommend the transcatheter bioprosthetic valve as the most suitable implantable heart valve for the patient.

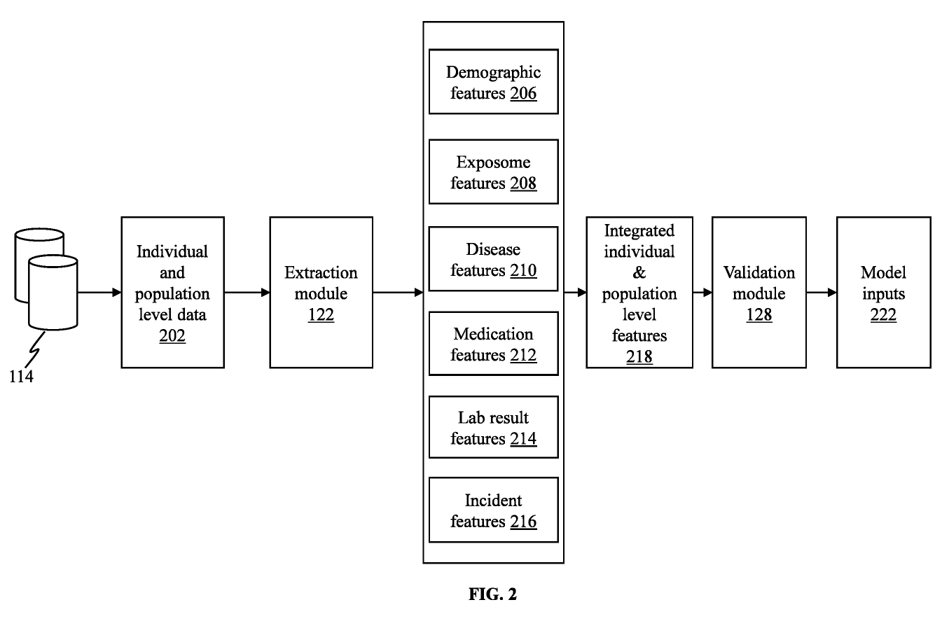

Referring to FIG. 2, in an example embodiment, a feature extraction and processing sequence executed on one or more electronic computing devices is disclosed. The patient health information 108A and the related clinical data 108B stored in the database 114 may be fed for extraction of corresponding features. The patient health information 108A and the related clinical data 108B may be in the form of query language databases, comma-separated value files (CSV), extensible markup language files (HTML), JavaScript object notation files (JSON) and others known to a person of ordinary skill in the art. A set of individual and population level data 202 may be obtained from the database 114 and may include without limitation, individual-level patient records including clinical, demographic, laboratory, medication, procedural, geographic and other patient-specific information, along with population-level data including census data, environmental exposure metrics, air pollution summary measures, and other community-level datasets, as well as population-level datasets indexed according to one or more geographical identifiers.

In an example embodiment, the individual-level data may be linked to the population-level data using a shared geographical identifier, such as a zip code, county code, or census tract identifier, such that the population-level information corresponding to a patient’s location may be associated with the patient’s record.

In an example embodiment, the extraction module 122 may be configured to receive the individual and population-level data 202 (raw data) and extract relevant data elements from the individual and population-level data 202. The extraction module 122 may align and merge population-level records with individual patient records based on the geographical identifier. For example, environmental exposure data recorded at a zip code level, such as average particulate matter 2.5 concentration values for a given year, may be associated with each patient residing within the corresponding zip code and added to the patient’s record. Further, the extraction module 122 may be configured to sort the raw data into a plurality of derived feature categories, including demographic features 206 such as age, sex, and income, as well as exposome features 208 which include records of environmental stressors, lifestyle, and external exposure factors. The categorization further includes disease features 210 representing identifiers for cardiovascular and heart-related diagnoses, medication features 212 consisting of standardized codes for pharmaceutical treatments and prescriptions, lab result features 214 containing numerical measurements and physiological parameters from clinical testing, and incident features 216 which document prior medical procedures or clinical events. Once categorized, these elements are compiled into a unified set of integrated individual and population level features 218 and passed to the validation module 128 that assesses whether clinical and population-level values fall within accepted statistical or physiological ranges. Following this validation, the system 100 generates model inputs 222, which serve as the final structured data, incorporating co-occurrence matrices and categorical heuristic classes, for use in model training and heart valve prediction.

Geographic and environmental data, such as weather, temperature, humidity, or pollution, are processed to generate the exposome features 208. This processing includes cross-referencing the patient’s geographic identifier with databases containing long-term environmental exposure information, such as concentration data for formaldehyde, acetaldehyde, or benzo-a-pyrene indexed by a geographic region. In an example embodiment, the server 110 performs an unsupervised machine-learning analysis to group geographic locations into exposure-based categories. The server 110 assigns a unique geographic exposure identifier to each exposure-based category and links each geographic location in the population-level environmental exposure data to a corresponding geographic exposure identifier.

In an example embodiment, the plurality of derived feature categories may be compiled into a unified set of integrated individual and population level features 218 including patient-specific clinical information with population-level demographic, environmental, and health-related data corresponding to the patient’s community or geographic environment.

In an example embodiment, the integrated individual and population feature 218 may be validated by the validation module 128. The validation module 128 may be configured to evaluate individual-level features by assessing whether reported values fall within known and accepted ranges for attributes such as age, concurrent diagnoses, laboratory results, and other clinical measures. In parallel, population-level features may be extracted and validated by determining whether the values fall within known statistical or demographic ranges. Upon validation, the individual-level features may be classified into subgroups based on characteristics including but not limited to age, sex, and income, while the population-level features may be grouped at geographic resolutions such as census tracts, counties, or zip codes using attributes such as age distribution, income, race, and related measures. In alternate example embodiments, subgrouping may be refined using one or more clustering techniques including K-means clustering and others known to a person skilled in the art, where the validated features may be organized into feature matrices to compute clusters at the individual and population-level.

Based on the validated and subgrouped features, the system 200 generates structured model inputs 222. To support the training of the machine learning model 126, the server 110 generates a conditional vector comprising at least the geographic exposure identifier and a valve category identifier. The server 110 then executes the CGAN using this conditional vector to generate tabular synthetic patient records. The conditional vector is a machine-readable data structure used to control the generation of the tabular synthetic patient records by constraining the CGAN based on the geographic exposure identifier and the valve category identifier. The valve category identifier represents a classification of an implantable heart valve based on its constitution and the specific implantation procedure. Further, the valve category identifier is distinct from the geographic exposure identifier and excludes geographic or environmental exposure information, ensuring the generative model learns the independent relationships between patient health, environmental context, and prosthesis type. These resulting consolidated vectors and co-occurrence matrices are provided to the machine learning model 126 for training, testing, and execution.

In an example embodiment, the exposome features 208 relating to where someone has lived over time may be calculated. Based on the locations and duration someone has lived in each geographical location, environmental exposures (such as particulate matter with a diameter of 205 micrometres or less) may be considered, even if that person no longer lives in that location. Each exposure may be weighted, where the weighting coefficient may be the fraction of time spent in a location divided by the total amount of time across all locations. The weighted exposure may be further used to calculate the final exposome measure for an individual.

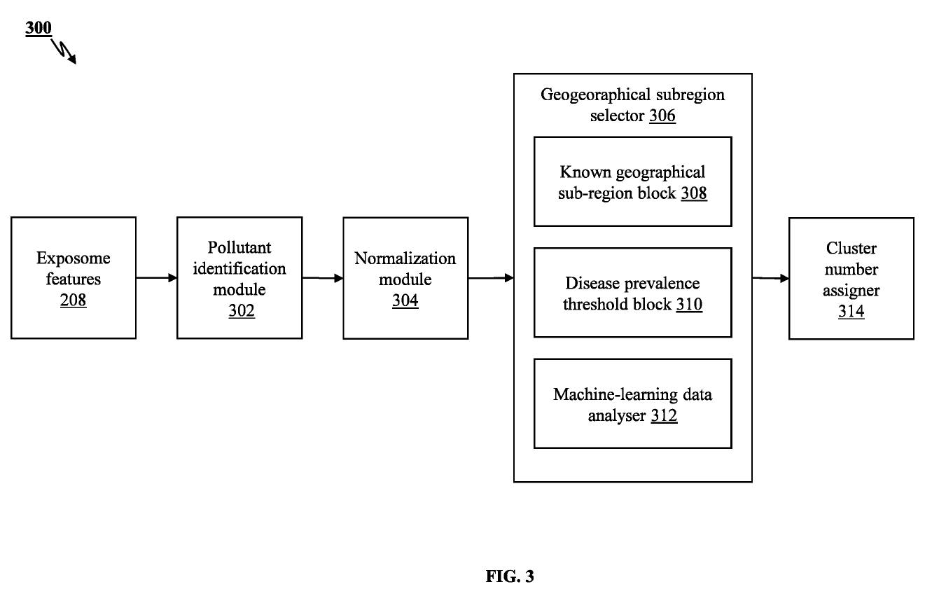

Referring to FIG. 3, in an example embodiment, a pollutant analysis sequence 300 integrated within the system 100 is disclosed. The exposome features 208 obtained from the extraction module 122 are processed to screen for and identify environmental pollutants associated with adverse cardiovascular and heart valve health outcomes. This screening targets pollutants known to increase risks for cardiac conditions, such as heart valve disease. For example, the system may identify aldehydes within the exposome features 208, as these pollutants are linked to increased risks of coronary heart disease.

In an example embodiment, the exposome features 208 may be provided to a pollutant identification module 302 configured to identify pollutant-related features from the received exposome data. The pollutant identification module 302 may access one or more pollutant reference databases, including but not limited to databases storing ambient aldehyde concentration values mapped to the geographic subregions. Based on such reference data, a normalization module 304 operating with the pollutant identification module 302 may be configured to normalize the exposome features to account for regional variability, measurement scale differences, and temporal factors, thereby generating normalized pollutant exposure measures suitable for subsequent analysis. For an example: If a patient lived in a coastal city with high humidity and a mountain region with low humidity, the normalization module 304 scales these environmental variables so that these variables can be compared fairly by the machine learning model without one scale overpowering the other.

In an embodiment, geographical subregion selector 306 is operatively coupled to the pollutant identification module 302 to identify areas with an elevated prevalence of coronary heart disease. The selector 306 includes a known geographical sub-region block 308 to reference historical disease data and a disease-prevalence threshold block 310 to determine if a subregion satisfies a predefined prevalence condition. Furthermore, a machine-learning data analyser 312 performs an unsupervised machine-learning analysis to group geographic locations into exposure-based categories based on shared environmental signatures. Upon conducting this analysis, a cluster number assigner 314 assigns a unique geographic exposure identifier to each identified category, where each geographic location in the population-level environmental exposure data is linked or mapped to its corresponding geographic exposure identifier. As a result, the system facilitates the indexing of the mapped residence locations within the patient-specific data stored on the user device 104. For an example: A “Cluster 001” identifier may be assigned to a group of geographic locations characterized by high industrial aldehyde concentrations. Any patient record indicating a residence history within these specific locations is then automatically tagged with the “Cluster 001” geographic exposure identifier to ensure environmental risks are captured.

In an alternate example embodiment, the system 300 employs these geographic exposure identifiers to calculate a location-weighted exposure score. For each patient, the server 110 identifies geographic locations associated with their residence history and retrieves the pollutant concentration values indexed by the geographic exposure identifiers for those locations. Further, the system 300 applies a residence-duration-based weighting to these values. For an example: For a patient who lived 8 years in “Cluster 1” (high formaldehyde) and 2 years in “Cluster 2” (low formaldehyde), the weighting coefficient for Cluster 1 is 0.8 (8/10 years). The final score is the sum of these duration-weighted values, ensuring the model prioritizes the environment where the patient spent the most time. The resulting cluster-based identifiers and weighted scores are propagated to individual-level training data. These data are transformed into structured model inputs (refer to model inputs 222) by combining them with patient clinical features to form co-occurrence matrices and categorical heuristic classes. These structured inputs are then used to train the ensemble model to satisfy the selection criterion of minimizing the difference between predicted patient lifespan and valve operational lifespan.

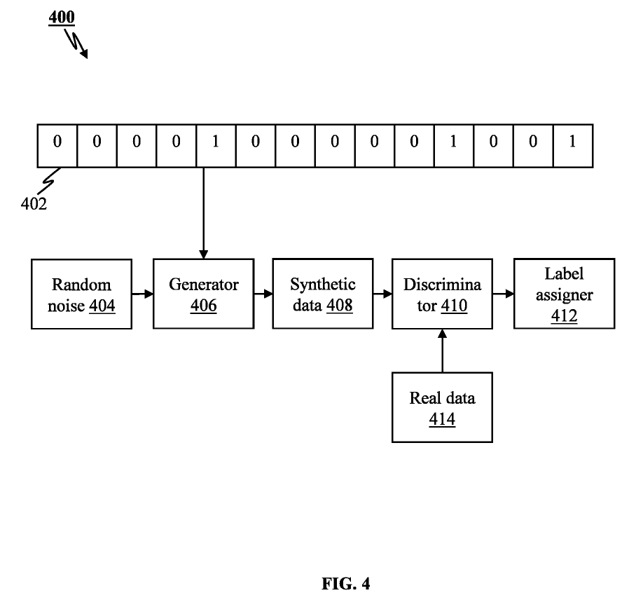

Referring to FIG. 4, in an example embodiment, the server 110 executes the Generative Adversarial Network (GAN) 400 to produce artificial patient records that mimic real-world clinical patterns through a structured, multi-step process. Initially, the server 110 encodes patient and environmental variables into a conditional vector 402, which serves as an encoded machine-readable representation of specific constraints. For example, the server 110 may transform categories such as a “High-Pollution Urban Zone” (geographic exposure identifier) and a “Transcatheter Tissue Valve” (valve category identifier) into a binary string that guides the generation process. Next, the generator 406 receives random noise 404 combined with these constraints from the conditional vector 402 to invent synthetic data 408. This generated data includes simulated patient identities, environmental histories, and heart valve-related clinical outcomes, such as a predicted operational lifespan.

Simultaneously, the discriminator 410 acts as a quality controller by comparing the synthetic data 408 against real data 414 sourced from verified clinical records. To enable learning, a label assigner 412 marks the real data 414 as “1” (as shown in FIG. 4) and the synthetic data 408 as “0”. Based on how well the discriminator 410 distinguishes between the two, the server 110 calculates a loss and adjusts the internal mathematical weights of the generator 406. Through repeated cycles, the internal mathematical weights of the generator 406 are adjusted until the synthetic records are statistically indistinguishable from real medical records.

Upon the CGAN 400 reaching convergence, the server 110 performs the step of generating tabular synthetic patient records. These records feature indexed data fields that represent patient identity information along with the geographic exposure context (via the geographic exposure identifier) and the heart valve classification context (via the valve category identifier). Further, these records include heart valve-related clinical outcome data, such as the predicted operational lifespan of the implant and its failure probability. The server 110 then proceeds with indexing the tabular synthetic patient records to form a synthetic clinical dataset, which is stored on the server. To ensure the system 100 is ready for local deployment, the server 110 performs the task of assigning a machine-learning parameter identifier to the predefined set of machine-learning parameters, such as optimized learning rates or tree depths. Finally, the process concludes with transmitting, from the server 110 to the user device 104, the synthetic clinical dataset together with the machine-learning parameter identifier. This transmission enables the user device 104 to locally train or execute the trained ensemble machine-learning model using the specific configuration and data context required for accurate heart valve recommendation.

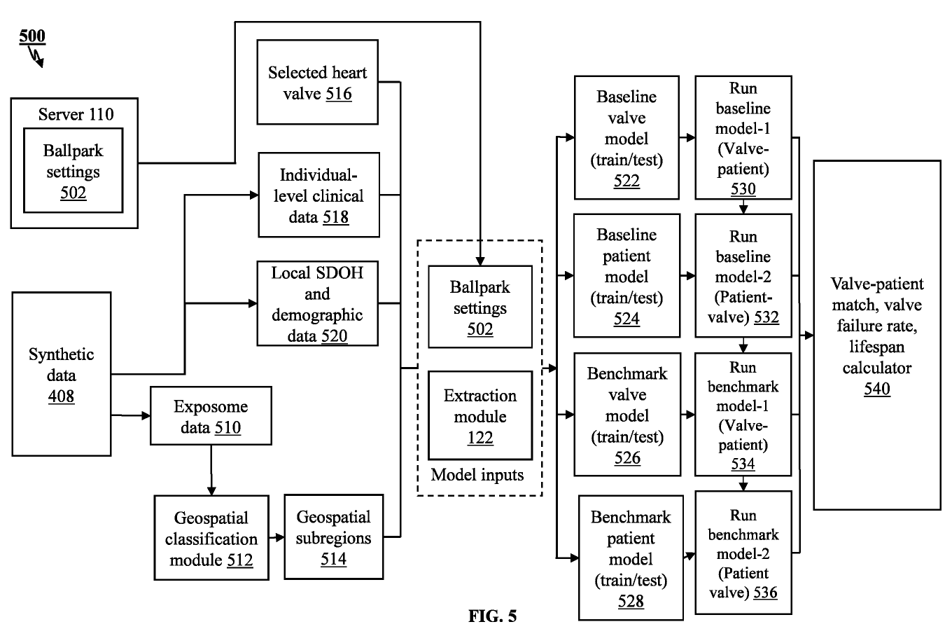

Referring to FIG. 5, a workflow 500 for training and testing a supervised ensemble machine-learning model 126 on a user device 104 is disclosed. The ensemble machine-learning model 126 may be configured to calculate a matching heart valve implant by minimizing the difference between a patient’s predicted remaining lifespan and the heart valve’s operational lifespan while ensuring the lowest failure and complication rates. The resulting matches are sorted from the highest to lowest probability of suitability for the patient.

In an example embodiment, the user device 104 receives ballpark settings 502 from the server 110 to configure the local machine learning environment. These settings include hyperparameter ranges, which define the configuration boundaries for the model, such as setting the maximum depth of a decision tree or the learning rate, to reduce the computational search space on the device. They also include train/test split ratios, which are used to divide the data into distinct sets for learning and evaluation, such as an 80/20 split where 80% of the data is used to train the model and 20% is reserved to test its predictive accuracy. These configurations are processed on the user device 104 alongside the synthetic data 408, individual-level clinical data 518, and local SDoH and demographic data 520. By performing these computations locally, the user device 104 functions as an edge computing node, enabling real-time, privacy-preserving model training and inference without the need to transmit sensitive patient clinical data back to the central server. The individual-level clinical data 518 consists of patient-specific medical records, including laboratory results, medication history, and documented procedures, while the local SDoH (Social Determinants of Health) and demographic data 520 includes information regarding a patient's socioeconomic status, education, and community characteristics that influence health outcomes. The extraction module 122 then identifies and pulls relevant features 206-216 from these combined data sources to create a structured input for the models. Simultaneously, the workflow 500 handles complex environmental information by processing exposome data 510 through a geospatial classification model 512. This model simplifies diverse geographic data into a set of unique integer values representing specific geospatial subregions 514, such as assigning a specific code to areas with similar pollution profiles. These integer identifiers are assigned to individuals within the synthetic data 408 and fed back into the extraction module 122. This integration ensures that the final features used by the models contain both the patient’s medical history and their specific regional environmental risk factors.

In an example embodiment, the system 500 may be configured to calculate a location-weighted exposure score by retrieving pollutant concentration values indexed by geographic exposure identifiers and correlating them with specific residence durations. For example, if a patient resided in “Subregion A” (high formaldehyde) for 10 years and “Subregion B” (low formaldehyde) for 10 years, the system 500 aggregates these duration-weighted values to determine the cumulative environmental impact on heart valve health. This calculated score is then processed as model input data alongside specific patient clinical parameters to train the supervised ensemble machine-learning model 126.

The system 100 employs an architecture consisting of two baseline ensemble models and two benchmark ensemble models. The baseline valve model 522 may be configured to identify an appropriate heart valve for a specific patient, while the baseline patient model 524 determines which patient profiles correspond to given heart valves. Further, the benchmark valve model 526 and benchmark patient model 528 may be configured with alternative hyperparameters or structural variations customized for specific regions, demographic profiles, or exposome classifications. These benchmark models may be evaluated against each other and the baseline models to validate predictive consistency, utilizing ballpark settings 502 to effectively reduce the computational search space on the user device 104. Further, the trained models may be executed for each valve category identifier to generate critical predictive outputs, including a predicted patient remaining lifespan value, a predicted valve operational lifespan value, and a predicted valve failure or complication probability. To ensure accuracy, the results from the run baseline model-1 530 (Valve-patient) and run baseline model-2 532 (Patient-valve) are compared against the original patient data to verify concordance. Similarly, outputs from run benchmark model-1 534 and run benchmark model-2 536 are processed to validate these predictions and confirm they align with verified clinical patterns.

In an example embodiment, a lifespan calculator 540 determines the difference between the patient's remaining lifespan and the predicted operational lifespan of a selected heart valve 516. The valve category identifiers are ordered based on this lifespan difference value and the predicted failure or complication probability, with the resulting ordered identifiers stored as output data on the user device 104. The validation module 128 assesses these results to determine whether a valve is a “good match” or a “poor match” based on defined concordance thresholds. Through edge-based validation, the validation module 128 enables the clinician to adjust thresholds and repeat analysis instantly on the user device 104, ensuring the heart valve recommendation is refined based on immediate clinical judgment. For instance, if the patient has a predicted 30-year remaining lifespan, the module ensures they are matched to a valve with at least 30 years of operational lifespan; a valve with a 70% predicted failure rate would be flagged as inappropriate compared to a valve with a 5% rate. The final validated outputs are encoded and transmitted for display on the user device 104, providing essential decision-support feedback to the user 102.

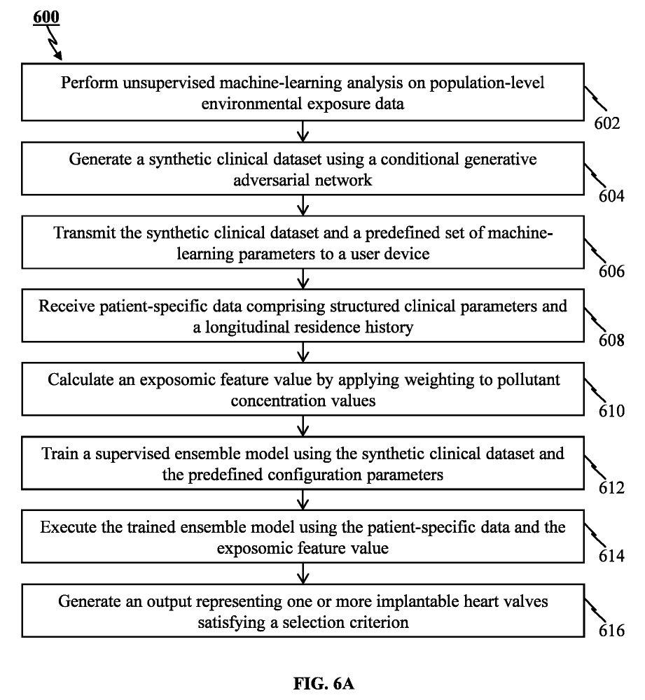

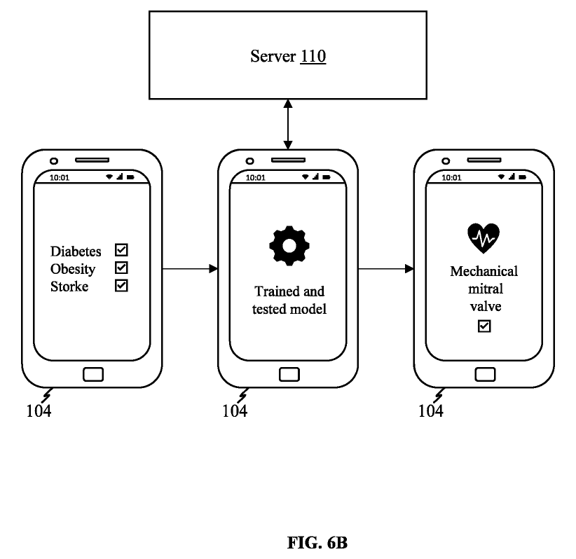

Referring to FIG. 6A, in an example embodiment, a method 600 for selecting an implantable heart valve for the patient is disclosed. The method 600 may be executed by the processor 130 (similar to processor 806) within the server 110, wherein each step of the method 600 may be performed by the processor 806 in accordance with instructions stored in the memory 810.

At step 602, upon receiving the patient health information 108A and the related clinical data 108B from the user 102 and storing in the database 114, the server 110 may be configured to perform an unsupervised machine-learning analysis on population-level environmental exposure data consisting of population-weighted atmospheric pollutant concentration values such that the server 110 may group geographic locations into exposure-based categories associated with cardiovascular risk.

For example, the server 110 may access population-weighted ambient concentration data for atmospheric pollutants including formaldehyde, acetaldehyde, and propionaldehyde at the county level across the United States.

The resulting exposure-based categories may include:
Exposure Region A: counties characterized by relatively high formaldehyde concentrations,
Exposure Region B: counties characterized by moderate concentrations of multiple aldehydes, and
Exposure Region C: counties characterized by relatively low concentrations of aldehydes.

At step 604, the server 110 may be configured to generate the synthetic clinical dataset using the CGAN configured to produce tabular patient data conditioned on at least one of an exposure-based geographic categories and a heart valve intervention category.

For example, the server 110 may be configured to generate the synthetic clinical dataset conditioned on the following inputs:
Exposure Category A: high aldehyde exposure region
Heart Valve Category 1: transcatheter aortic valve prosthesis

At step 606, the server 110 may be configured to transmit the synthetic clinical dataset and a predefined set of machine-learning parameters which includes clinical, demographics, address data (which can be used to infer SDoH and exposome information), or a database of patient information and features, or information provided by another electronic source to the user device 104.

At step 608, patient-specific data comprising structured clinical parameters and a longitudinal residence history may be received by the user device 104. The structured clinical parameters may include demographic information, clinical data, physiological measurements, laboratory results, comorbidity indicators, and procedural risk metrics relevant to cardiovascular assessment and implantable heart valve selection. The longitudinal residence history may include a plurality of geographic locations associated with the patient and corresponding residence durations for each location. Each geographic location may be represented using standardized geographic identifiers, including, but not limited to, counties, zip codes, census tracts, or other administrative regions. The residence durations may represent cumulative time spent by the patient at each geographic location over a defined period.

For example, the user device 104 may receive the following patient-specific data:
Structured clinical parameters:
   Age: 69 years
   Sex: female
   Diagnosis: aortic valve regurgitation
   Left ventricular ejection fraction: 55%
   Valve annulus diameter: 22 mm
   Comorbidities: diabetes, atrial fibrillation
Longitudinal residence history:
   Zip Code 10001: 12 years
   Zip Code 30303: 8 years

At step 610, the user device 104 may calculate the exposomic feature value for the patient by applying residence-duration-based weighting to pollutant concentration values associated with each geographic location in the patient’s longitudinal residence history. The longitudinal residence history may include a plurality of geographic locations and corresponding durations of residence, as shown in step 608. Further, for each geographic location, the user device 104 may determine one or more environmental exposure values, including ambient concentrations of pollutants known to adversely affect cardiovascular or heart valve health. The user device 104 computes a weighting coefficient for each geographic location based on the fraction of total residence time spent at that location relative to the total cumulative residence duration across all locations.

For example, the patient's longitudinal residence history processed by the user device 104 may include the following:
Zip Code 10001:
   Duration: 12 years
   Ambient methanol concentration: 6 µg/m³
Zip Code 30303:
   Duration 8 years
   Ambient methanol concentration: 4 µg/m³
The total residence duration across all locations is 20 years. The weighting coefficients are therefore:
   Zip Code 10001: 12 / 20 = 0.6
   Zip Code 30303: 8 / 20 = 0.4
The user device 104 may then calculate a time-weighted exposomic feature value for methanol exposure as 5.2 ug/m3.

At step 612, the user device 104 may be configured to train the ensemble machine-learning model using the synthetic clinical dataset and predefined configuration parameters. The supervised ensemble machine-learning model may be selected from one or more logistic regression, random forest models, gradient-boosted decision tree models, support vector machines, artificial neural networks, and elastic net regression. The synthetic clinical dataset may include tabular patient records generated using a generative adversarial network and may comprise clinical features, exposomic feature values, geographic exposure categories, heart valve intervention categories, and associated outcome labels.

At step 614, the user device 104 may be configured to execute the trained supervised ensemble machine-learning model using patient-specific data and the calculated exposomic feature value. The patient-specific data may include structured clinical parameters and longitudinal residence history-derived features, as previously received and processed. The exposomic feature value may represent a time-weighted cumulative environmental exposure associated with the patient.

Upon execution, the trained ensemble machine-learning model may generate the output data representing one or more implantable heart valves that satisfy a predefined selection criterion. The selection criterion may include minimizing a difference between a predicted remaining lifespan of the patient and a predicted operational lifespan of the implantable heart valve, while simultaneously reducing a predicted probability of valve failure, degeneration, or procedure-related complications. The predicted remaining lifespan of the patient and the predicted operational lifespan of each candidate implantable heart valve may be generated as part of the model output or derived from model-predicted risk scores and durability estimates. For example, based on the inputs, the machine-learning model may generate predictions for multiple implantable heart valves, including:
Valve A (Transcatheter valve):
   Predicted patient remaining lifespan: 14 years
   Predicted valve operational lifespan: 13 years
   Predicted complication probability: 6%
Valve B (Surgical bioprosthetic valve):
   Predicted patient remaining lifespan: 14 years
   Predicted valve operational lifespan: 9 years
   Predicted complication probability: 12%
Based on the selection criterion favoring a reduced difference between patient lifespan and valve lifespan and a lower predicted complication probability, Valve A may be recommended as the implantable heart valve for the patient.

Referring to FIG. 6B, in an example edge computing embodiment, an exemplary execution of a trained and tested ensemble machine-learning model on the user device 104 using the application platform 106 is illustrated. The patient health information 108A may be first obtained via the application platform 106 through one or more user interface elements implemented on the user device 104, including, but not limited to, checkboxes, voice input mechanisms, free-text entry fields, buttons, menus, and other standard software interface components. The patient health information 108A may include structured and unstructured clinical data, demographic information, and other patient-specific inputs relevant to heart valve selection.

The application platform 106 may be configured to process the received patient health information 108A to compute feature values suitable for input into the trained and tested ensemble machine-learning model. Further, the application platform 106 running on the user device 104 may transmit a request to the server 110 for a clinical synthetic dataset. In response to the request, the server 110 may generate synthetic data 408 from real clinical data 414 using a generative adversarial network and may transmit the generated synthetic data 408 to the user device 104. The synthetic dataset may be used to support model execution, calibration, or validation without exposing identifiable patient information.

Subsequently, feature values derived from the patient health information 108A, including clinical, demographic, geospatial, exposomic, and other relevant attributes, may be provided as inputs to the trained and tested ensemble machine-learning model executed on the user device 104. Upon execution, the model may analyze the input features in conjunction with learned patterns from the synthetic dataset to generate one or more outputs representing matching implantable heart valve options corresponding to the patient-specific feature profile. The calculated results may then be presented through the application platform 106 to support clinical decision-making.

In an alternate example embodiment, in connection with the exceptions that may arise during execution of the ensemble machine-learning model. In certain cases, statistical parameters generated for two or more candidate implantable heart valves, such as odds ratios, confidence intervals, or predicted risk scores, may be statistically equivalent or fall within a predefined equivalence threshold, thereby making automated selection between the valves indeterminate. When such an exception is detected, the system 100 may present an interactive interface to the user 102 requesting confirmation or review of key patient information, including clinical parameters, anatomical measurements, or exposomic features. Upon confirmation or update of the information, the ensemble machine-learning model may be re-executed using verified or supplemented data. A subsequent report indicating that the model has been re-run may then be generated and presented to the user 102 together with any changes in predicted valve suitability, statistical parameters, or explanatory features derived from the explanatory analysis. If the exception condition persists following re-execution of the model, the system 100 may present the unresolved exception to the user 102 and provide options to consult a clinician, enter additional patient-specific information, or manually select among the candidate implantable heart valves.

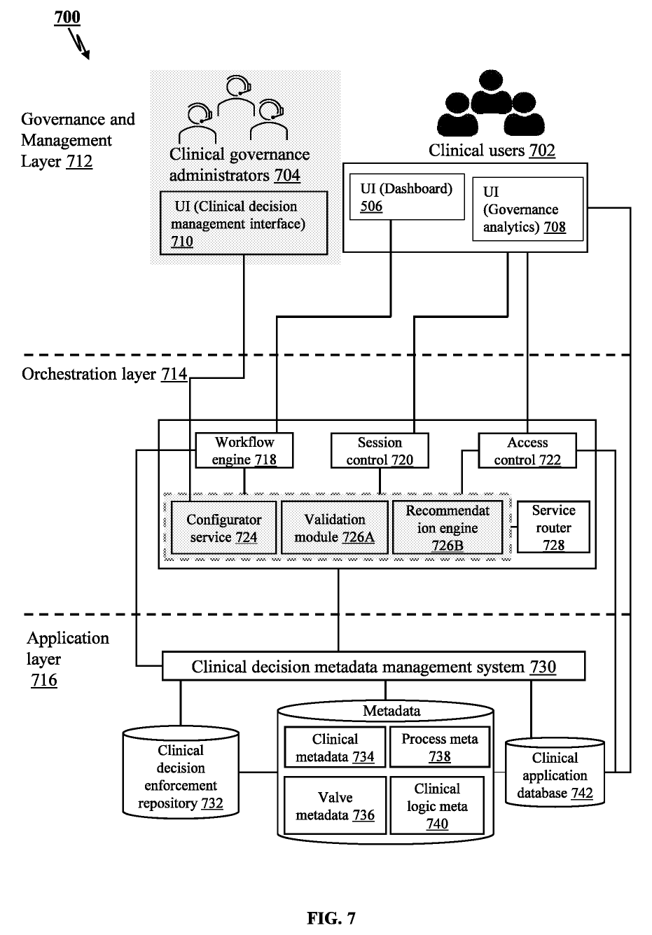

Referring to FIG. 7, in an example embodiment, a computing environment system 700 for implementing the system 100 for selecting the implantable heart valve for patients within the application platform 106 is disclosed. The system 700 may be configured to provide a cloud-based clinical decision-support platform for selecting an appropriate implantable heart valve for the patient. The system 700 may support interaction between clinical users 702 (e.g., cardiologists, cardiac surgeons, clinical staff) and clinical governance administrators 704 (e.g., system operators, clinical data stewards, regulatory or quality assurance personnel). The architecture may be implemented using modular components that collectively enable secure access control, configurable clinical workflows, patient-specific data processing, machine-learning-based evaluation, and persistent management of clinical and configuration data. The system 700 may support core functionalities including processing patient-specific clinical and exposomic data, executing trained machine-learning models, evaluating candidate implantable heart valves, and generating valve selection recommendations based on predefined clinical criteria.

In an example embodiment, the system 700 includes a clinical decision management interface 710 at a governance and management layer 712, configured as a system-facing user interface for clinical governance administrators 704. The clinical decision management interface 710 may enable clinical governance administrators 704 to configure valve selection parameters, define evaluation criteria, manage clinical validation rules, set model configuration parameters, and monitor system performance and compliance metrics. The clinical decision management interface 710 may further allow administrative override of selection thresholds, update of valve intervention categories, and management of workflow policies used in patient-specific evaluation.

In an example embodiment, patient-specific data and administrative requests may be received by the system 700 through a secure access layer configured to enforce authentication and authorization policies. A user management and access control component may ensure that only credentialed clinical users and authorized administrators can access protected system resources, submit patient data, configure workflows, or review valve selection outputs. Requests may be routed through a centralized request handling component that ensures system availability, load balancing, and secure communication across system modules.

In an example embodiment, the system 700 may include an orchestration layer 714 configured to manage coordination among modular services involved in the clinical decision workflow. Within the orchestration layer 714, independently deployable services may include a configuration service 724, a clinical validation module 726A, and a recommendation engine 726B. The configuration service 724 may manage intervention definitions, selection criteria, and model parameters. The clinical validation module 726A may verify correctness, completeness, and clinical compliance of patient-specific and exposomic inputs. The recommendation engine 726B may evaluate candidate implantable heart valves by applying trained ensemble machine-learning models to generate suitability scores, predicted valve durability, and complication risk estimates.

In an example embodiment, the system 700 may include a workflow engine 718 configured to define and execute clinical decision workflows for selecting an appropriate implantable heart valve for the patient. The workflow engine 718 may orchestrate sequential and dependent processing steps, including intake of patient-specific clinical data, validation of inputs, calculation of exposomic feature values, execution of trained machine-learning models, and generation of valve selection recommendations. The orchestration layer 714 may further include a session control 720 and an access control 722. The session control 720 may manage user sessions associated with clinicians and administrative users, maintain session state, and ensure continuity of patient-specific evaluation workflows across multiple interactions. The access control 722 may enforce authentication and authorization policies by verifying user credentials, determining user roles, and restricting access to clinical data, configuration settings, and decision outputs based on predefined permission levels.

In an example embodiment, the system 700 may include an application layer 716 which provides persistent storage, governance, and enforcement support for the implantable heart valve selection workflows. The layer may further include a clinical decision metadata management system 730 configured to store and manage structured metadata associated with clinical decision-making. The metadata may include clinical metadata 734 defining patient-related parameters and clinical concepts, valve metadata 736 describing implantable heart valve intervention characteristics, process metadata 738 defining workflow steps and execution logic, and clinical logic metadata 740 representing decision rules, thresholds, and evaluation criteria.

Further, a clinical decision enforcement repository 732 may be configured store executable rules, policies, and constraints used to enforce clinical validation, safety requirements, and selection criteria during valve evaluation. The application layer 714 may also include a clinical application database 742 configured to store application-specific data such as patient records, exposomic feature values, evaluation outputs, audit logs, and historical valve selection results.

Accordingly, the computing environment described herein provides a flexible, scalable, and clinically focused platform for implementing a patient-specific implantable heart valve selection system. The architecture supports secure ingestion of clinical and exposomic data, configurable and auditable decision workflows, execution of machine-learning based evaluation models, and consistent generation of explainable valve selection recommendations. The modular design enables future enhancements, including integration of additional clinical data sources, updated exposure models, alternative machine-learning techniques, and evolving clinical guidelines, without departing from the scope of the present disclosure.

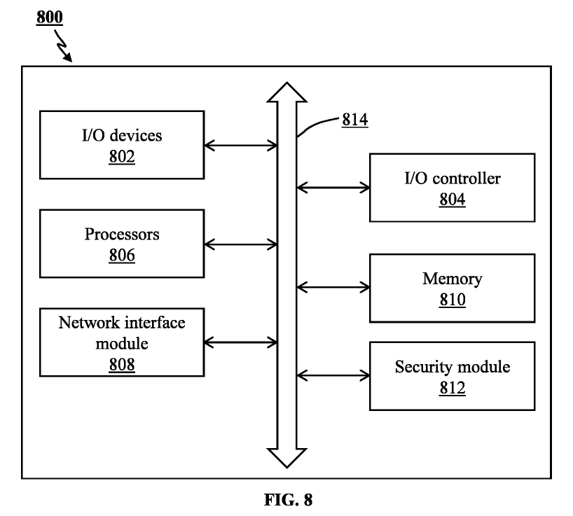

Referring to FIG. 8, in an example embodiment, a system architecture 800 for the server 110 on which an example embodiment of the implantable heart valve selection system may be executed is disclosed. The server 110 may be implemented as a physical or virtual computing device, including, but not limited to, a rack-mounted server, a cloud-based instance, a workstation, a hospital on-premises computing node, or a hybrid deployment environment. The server 110 may be operatively configured to perform one or more functions described herein, including, but not limited to, receiving patient-specific clinical inputs, processing anatomical and physiological data, computing suitability and compatibility scores for a plurality of implantable heart valves, executing clinical decision logic, and generating structured recommendations for selecting the implantable heart valve for the patient.

In an example embodiment, the server 110 may include one or more input/output (I/O) devices 802, an input/output controller 804, one or more processors 806, a network interface module 808, a memory 810, and a security module 812, all communicatively coupled via a system bus 814. The system bus 814 may include a memory bus, a peripheral bus, or a combination thereof, and may utilize standardized communication protocols including, but not limited to, PCIe, I²C, or SPI.

In an example embodiment, the input/output devices 802 may include hardware interfaces for receiving clinician input and presenting system output. The I/O devices 802 may include, for example, a keyboard, mouse, touchscreen, monitor, or audio interface, thereby enabling interaction with the system for entry of patient data, imaging references, procedural constraints, validation inputs, system configuration, visualization of recommendations, or clinical review. In certain example embodiments, the I/O devices 802 may further include interfaces to medical peripherals or diagnostic systems for importing patient measurements or examination results relevant to heart valve selection.

In an example embodiment, the input/output controller 804 may be configured to manage data flow between the peripheral hardware and internal system components. The input/output controller 804 may implement standard interface protocols (e.g., USB, HDMI, UART) and provide functionality for timing control, interrupt handling, and access arbitration. In certain example embodiments, the input/output controller 804 may additionally enforce access permissions and session-level protocols to support secure and authorized interaction between authorized clinical users and the system.

In an example embodiment, the one or more processors 806 (similar to processor 130) may include, without limitation, central processing units (CPUs), graphics processing units (GPUs), neural processing units (NPUs), or other heterogeneous computing architectures. The one or more processors 806 may be configured to execute instructions to perform operations associated with method 600, including processing patient-specific anatomical parameters, evaluating procedural constraints, matching patient data against valve specifications, computing compatibility and risk scores, and generating ranked recommendations for one or more implantable heart valves suitable for the patient.

In an example embodiment, the system 800 may include a network interface module 808 configured to enable communication between the system and external devices, systems, or services over one or more networks. The network interface module 808 may include wired and/or wireless communication components, including, without limitation, Ethernet adapters, Wi-Fi transceivers, or cellular radios. The network interface module 808 may support standard networking protocols including, but not limited to, TCP/IP, HTTP/HTTPS, REST, and HL7 or FHIR-based healthcare communication protocols, thereby enabling secure exchange of patient data, valve specification data, and clinical guidelines relevant to implantable heart valve selection.

In an example embodiment, the memory 810 may include both volatile and non-volatile memory components. The volatile memory may include random access memory (RAM) for executing real-time data processing and clinical evaluation tasks, while the non-volatile memory may include solid-state drives (SSDs), hard disk drives (HDDs), or other persistent storage media for maintaining patient records, valve specification datasets, compatibility rules, risk models, scoring parameters, and configuration templates. The memory 810 may further store executable instructions that, when executed by the one or more processors 806, cause the system 800 to perform operations of method 600, including selection, and recommendation of implantable heart valves based on patient-specific criteria.

In an example embodiment, the security module 812 may be configured to ensure the integrity, confidentiality, and regulatory compliance of data processed within the system 100. The security module 812 may implement security functions including, but not limited to, data encryption, clinician authentication, role-based access control, audit logging, and secure data transmission. These functions serve to safeguard sensitive patient information, clinical decision data, and valve selection outputs, while maintaining compliance with applicable healthcare data protection regulations.

The disclosed system is applicable to a wide range of clinical environments and healthcare platforms in which accurate and explainable selection of an implantable heart valve provides significant clinical value. Conventional approaches to valve selection often rely on manual interpretation of guidelines, fragmented patient data, and subjective judgment, which may increase procedural risk or variability in outcomes. The system described herein addresses these limitations by systematically integrating patient-specific anatomical and clinical data with structured valve specifications and decision logic to support consistent and informed valve selection.

Additionally, the system 100 improves upon existing clinical decision-support methods by applying data-driven evaluation, rule-based reasoning, and compatibility scoring to assist clinicians in selecting an implantable heart valve tailored to the patient. By executing method 600, the system 100 reduces cognitive burden on clinicians, enhances transparency of selection rationale, supports scalable deployment across healthcare institutions, and promotes improved procedural planning and patient outcomes without reliance on video generation or multimedia explanation components.

Although example embodiments of the present disclosure have been described with reference to illustrative implementations, it will be understood by those skilled in the art that numerous modifications, substitutions, variations, and rearrangements of components may be made without departing from the broader spirit and scope of system 100. Accordingly, the foregoing description and accompanying drawings are to be interpreted in an illustrative rather than a limiting sense, with the scope of the disclosure being defined by the appended claims and their equivalents.

Many alterations and modifications of the present disclosure will no doubt become apparent to a person of ordinary skill in the art after having read the foregoing description. It is to be understood that the phraseology or terminology employed herein is for the purpose of description and not of limitation. It is to be understood that the description above contains many specifications; these should not be construed as limiting the scope of the disclosure but as merely providing illustrations of some of the example embodiments of this disclosure. Thus, the scope of the disclosure should be determined by the appended claims and their legal equivalents rather than by the examples given.

What is claimed is:

1. A computer-implemented method for selecting a heart valve intervention for a patient using a distributed calculating system comprising a computer server and an electronic user device, the method comprising:
performing, by the server, unsupervised machine-learning analysis on a population-level environmental exposure data comprising population-weighted atmospheric pollutant concentration values to group geographic locations into exposure-based geographic categories associated with cardiovascular risk;
generating, by the server, a synthetic clinical dataset using a generative adversarial network configured to produce tabular patient data conditioned on at least one of:
an exposure-based geographic category; and
a heart valve prosthesis category;
transmitting, by the server, the synthetic clinical dataset and a predefined set of machine-learning parameters to the user device;
receiving, by the user device, patient-specific data comprising structured clinical parameters and a longitudinal residence history including multiple geographic locations and corresponding residence durations;
calculating, by the user device, an exposomic feature value by applying residence-duration-based weighting to pollutant concentration values associated with each geographic location in the longitudinal residence history;
training and testing, by the user device, a supervised ensemble machine-learning model including gradient-boosted decision tree models and random forest models using the synthetic clinical dataset and predefined configuration parameters; and
executing, by the user device, the trained and tested supervised ensemble machine-learning model using the patient-specific data and the exposomic feature value to generate an output representing one or more implantable heart valves satisfying a selection criterion comprising a reduced difference between a predicted remaining lifespan of the patient and a predicted operational lifespan of the implantable heart valve, together with a reduced predicted valve failure or complication probability.

2. The method according to claim 1, further comprising:
assigning, by the server, a unique geographic exposure identifier to each exposure-based geographic category; and
linking each geographic location in the population-level environmental exposure data to a corresponding geographic exposure identifier.

3. The method according to claim 2, further comprising:
generating, by the server, a conditional vector comprising at least the corresponding geographic exposure identifier and a valve category identifier; and
executing the generative adversarial network using the conditional vector to generate tabular synthetic patient records, wherein:
the conditional vector is a machine-readable data structure used to control generation of the tabular synthetic patient records by constraining the generative adversarial network based on the geographic exposure identifier and the valve category identifier; and
the valve category identifier represents a classification of an implantable heart valve based on valve constitution and implantation procedure, the valve category identifier being distinct from the geographic exposure identifier and excluding geographic or environmental exposure information.

4. The method according to claim 3, further comprising:
generating the tabular synthetic patient records having indexed data fields that represent:
patient identity information along with geographic exposure context and heart valve classification context; and
heart valve related clinical outcome data;
indexing the tabular synthetic patient records to form a synthetic clinical dataset stored on the server;
assigning a machine-learning parameter identifier to the predefined set of machine-learning parameters; and
transmitting, from the server to the user device, the synthetic clinical dataset together with the machine-learning parameter identifier.

5. The method according to claim 4, further comprising:
receiving, by the user device, the longitudinal residence history;
mapping each residence location in the longitudinal residence history to the geographic exposure identifier; and
indexing the mapped residence locations within the patient-specific data stored on the user device.

6. The method according to claim 5, further comprising:
retrieving pollutant concentration values indexed by the geographic exposure identifiers;
correlating each pollutant concentration value with a residence duration; and
calculating a location-weighted exposure score by aggregating duration-weighted pollutant concentration values.

7. The method according to claim 6, further comprising:
training the supervised ensemble machine-learning model using the synthetic clinical dataset indexed by the valve category identifiers; and
processing the patient clinical parameters and the location-weighted exposure score as model input data.

8. The method according to claim 7, further comprising:
executing the trained and tested supervised ensemble machine-learning model for each valve category identifier; and
calculating a predicted patient remaining lifespan value, a predicted valve operational lifespan value, and a predicted valve failure or complication probability.

9. The method according to claim 8, further comprising:
calculating a lifespan difference value between the predicted patient remaining lifespan value and the predicted valve operational lifespan value;
ordering the valve category identifiers based on the lifespan difference value and the predicted valve failure or complication probability;
storing the ordered valve category identifiers as output data on the user device.

10. A system for selecting a heart valve intervention for a patient using a distributed calculating system, the system comprising:
a server;
a user device;
one or more processors; and
a non-transitory memory communicatively coupled to the one or more processors, wherein the one or more processors are configured to execute instructions stored in the non-transitory memory, the instructions causing the one or more processors to:
perform, by the server, unsupervised machine-learning analysis on a population-level environmental exposure data comprising population-weighted atmospheric pollutant concentration values to group geographic locations into exposure-based categories associated with cardiovascular risk;
generate, by the server, a synthetic clinical dataset using a generative adversarial network configured to produce tabular patient data conditioned on at least one of:
an exposure-based geographic category; and
a heart valve prosthesis category;
transmit, by the server, the synthetic clinical dataset and a predefined set of machine-learning parameters to the user device;
receive, by the user device, patient-specific data comprising structured clinical parameters and a longitudinal residence history including multiple geographic locations and corresponding residence durations;
calculate, by the user device, an exposomic feature value by applying residence-duration-based weighting to pollutant concentration values associated with each geographic location in the longitudinal residence history;
train, by the user device, a supervised ensemble machine-learning model selected from gradient-boosted decision tree models and random forest models using the synthetic clinical dataset and predefined configuration parameters; and
execute, by the user device, the trained and tested supervised ensemble machine-learning model using the patient-specific data and the exposomic feature value to generate an output representing one or more implantable heart valves satisfying a selection criterion comprising a reduced difference between a predicted remaining lifespan of the patient and a predicted operational lifespan of the implantable heart valve, together with a reduced predicted valve failure or complication probability.

11. The system according to claim 10, wherein the one or more processors are configured to:
assign, by the server, a unique geographic exposure identifier to each exposure-based category; and
link each geographic location in the population-level environmental exposure data to a corresponding geographic exposure identifier.

12. The system according to claim 11, wherein the one or more processors are configured to:
generate, by the server, a conditional vector comprising at least the corresponding geographic exposure identifier and a valve category identifier; and
execute the generative adversarial network using the conditional vector to generate tabular synthetic patient records, wherein:
the conditional vector is a machine-readable data structure used to control generation of the tabular synthetic patient records by constraining the generative adversarial network based on the geographic exposure identifier and the valve category identifier; and
the valve category identifier represents a classification of a heart valve intervention based on valve constitution and implantation procedure, the valve category identifier being distinct from the geographic exposure identifier and excluding geographic or environmental exposure information.

13. The system according to claim 12, wherein the one or more processors are configured to:
generate tabular synthetic patient records having indexed data fields that represent:
patient identity information along with geographic exposure context and heart valve classification context; and
heart valve related clinical outcome data;
index the tabular synthetic patient records to form a synthetic clinical dataset stored on the server;
assign a machine-learning parameter identifier to the predefined set of machine-learning parameters; and
transmit, from the server to the user device, the synthetic clinical dataset together with the machine-learning parameter identifier.

14. The system according to claim 13, wherein the one or more processors are configured to:
receive, by the user device, the longitudinal residence history;
map each residence location in the longitudinal residence history to the geographic exposure identifier; and
index the mapped residence locations within the patient-specific data stored on the user device.

15. The system according to claim 14, wherein the one or more processors are configured to:
retrieve pollutant concentration values indexed by the geographic exposure identifiers;
correlate each pollutant concentration value with a residence duration; and
calculate a location-weighted exposure score by aggregating duration-weighted pollutant concentration values.

16. The system according to claim 15, wherein the one or more processors are configured to:
train the supervised ensemble machine-learning model using the synthetic clinical dataset indexed by valve category identifiers; and
process the patient clinical parameters and the location-weighted exposure score as model input data.

17. The system according to claim 16, wherein the one or more processors are configured to:
execute the trained and tested supervised ensemble machine-learning model for each valve category identifier; and
calculate a predicted patient remaining lifespan value, a predicted valve operational lifespan value, and a predicted valve failure or complication probability.

18. The system according to claim 17, wherein the one or more processors are configured to:
calculate a lifespan difference value between the predicted patient remaining lifespan value and the predicted valve operational lifespan value;
order valve category identifiers based on the lifespan difference value and the predicted valve failure or complication probability; and
store the ordered valve category identifiers as output data on the user device.

19. A non-transitory computer-readable medium having stored thereon, computer- executable instructions which, when executed by a computer, cause the computer to execute operations, the operations comprising:
performing, by the server, unsupervised machine-learning analysis on a population-level environmental exposure data comprising population-weighted atmospheric pollutant concentration values to group geographic locations into exposure-based categories associated with cardiovascular risk;
generating, by the server, a synthetic clinical dataset using a generative adversarial network configured to produce tabular patient data conditioned on at least one of:
an exposure-based geographic category; and
a heart valve prosthesis category;
transmitting, by the server, the synthetic clinical dataset and a predefined set of machine-learning parameters to the user device;
receiving, by the user device, patient-specific data comprising structured clinical parameters and a longitudinal residence history including multiple geographic locations and corresponding residence durations;
calculating, by the user device, an exposomic feature value by applying residence-duration-based weighting to pollutant concentration values associated with each geographic location in the longitudinal residence history;
training, by the user device, a supervised ensemble machine-learning model selected from gradient-boosted decision tree models and random forest models using the synthetic clinical dataset and predefined configuration parameters; and
executing, by the user device, the trained and tested supervised ensemble machine-learning model using the patient-specific data and the exposomic feature value to generate an output representing one or more implantable heart valves satisfying a selection criterion comprising a reduced difference between a predicted remaining lifespan of the patient and a predicted operational lifespan of the implantable heart valve, together with a reduced predicted valve failure or complication probability.
